(19) United States
(12) Patent Application Publication
(10) Pub. No.: US 2003/0022912 A1
(43) Pub. Date: Jan. 30, 2003

(54) Rapid-onset medicament for treatment of sexual dysfunction

(76) Inventors: Alice C. Martino, Kalamazoo, MI (US); Vincent E. McCurdy, Portage, MI (US); Steven A. Pierman, Portage, MI (US); Joseph P. Reo, Kalamazoo, MI (US); Praveen Tyle, Kalamazoo, MI (US); Sy-Juen Wu, Portage, MI (US)

Correspondence Address: Pharmacia Corporation, Corporate Patent Department, 800 N. Lindbergh Boulevard-04B, St. Louis, MO, 63167, US

(21) Appl. No.: 10/072,368

(22) Filed: Feb. 7, 2002

Related U.S. Application Data
(60) Provisional application No. 60/267,519, filed on Feb. 8, 2001.

(51) Int. Cl. A61K 31/4745

(52) U.S. Cl. 514/292

(57) ABSTRACT

A rapid-onset pharmaceutical composition is provided, useful for treatment of sexual dysfunction, stimulation of sexual activity and enhancement of sexual desire, interest and performance in men and women. The composition is a dosage form comprising (a) a therapeutically or sexual-stimulatorily effective amount of a therapeutic agent having a molecular weight, excluding counterions, not greater than 250, and (b) at least one pharmaceutically acceptable excipient; and is adapted for delivery by a route of administration that entails interaction with the organs of taste yet has acceptable organoleptic properties. Illustrative therapeutic agents useful in dosage forms of the invention are compounds having the formula

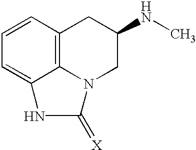

wherein X is O or S; and pharmaceutically acceptable salts thereof.

This application claims priority of U.S. Provisional Application Serial No. 60/267,519, filed on Feb. 8, 2001.

### FIELD OF THE INVENTION

The present invention relates to rapid-onset pharmaceutical compositions containing a water-soluble drug useful in treatment of male and female sexual dysfunction, to processes for preparing such compositions and to methods of treatment comprising administering such compositions to a subject by an appropriate route of administration. The term “rapid-onset” applied to a composition or dosage form herein means that a therapeutic effect is achievable within a short period of time, for example less than about 1 hour, following administration.

### BACKGROUND OF THE INVENTION

Orally administered therapies for sexual dysfunction, in particular for male erectile disorder, are well known. See for example Gingell & Lockyer (1999), “Emerging pharmacological therapies for erectile dysfunction”, Expert Opinion on Therapeutic Patents 9, 1689-1696. Drugs in use or in development include phosphodiesterase type 5 (PDE5) inhibitors, e.g., sildenafil citrate, available under the trademark Viagra® of Pfizer, cyclic AMP activators, α-adrenergic antagonists, e.g., yohimbine, and dopaminergic agonists, e.g., apomorphine.

International Patent Publication No. WO 00/40226 discloses compounds useful in treating sexual dysfunction in men and women, these compounds being of formula (I)

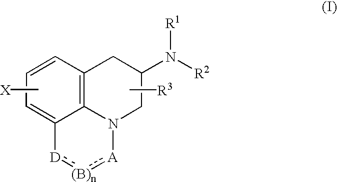

or pharmaceutically acceptable salts thereof, wherein

R¹,R² and R³ are the C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl;

A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;

B is CH, CH₂, CHF, CHCl, CHBr, CHI, C═O, N, NH or NCH₃, and n is 0 or 1; and

D is CH, CH₂, CHF, CHCl, CHBr, CHI, C═O, O, N, NH or NCH₃;

with various provisos indicated therein. WO 00/40226 further contemplates prescription of the drug (R)-5,6-dihydro-5-(methylamino)-4H-imidazo[4,5-ij]-quinolin-2(1H)-one (Z)-2-butenedioate (1:1) to male and female subjects at a dose of 1-3 mg, to be taken 0.5-1 h before engaging in sexual activity, and indicates that at such a dose and timing of administration the drug is therapeutically effective. No information is provided as to the route of administration or nature of dosage form.

The class of compounds proposed for treatment of sexual dysfunction in WO 00/40226 was earlier disclosed in U.S. Pat. No. 5,273,975 to Moon et al. to have therapeutically useful central nervous system activity. Above-cited International Patent Publication No. WO 00/40226 and U.S. Pat. No. 5,273,975 are incorporated herein by reference. Certain compounds of the above class are the subject of a paper by Heier et al. (1997), “Synthesis and biological activities of (R)-5,6-dihydro-N,N-dimethyl-4H-imidazo[4,5,1-ij]quinolin-5-amine and its metabolites”, J. Med. Chem. 40, 639-646.

International Patent Publication No. WO 99/16442, incorporated herein by reference, discloses a sustained-release tablet formulation of (R)-5,6-dihydro-5-(methylamino)-4H-imidazo[4,5-ij]-quinolin-2(1H)-one (Z)-2-butenedioate (1:1) for treatment of Parkinson's disease.

In spite of the availability of sildenafil citrate, apomorphine and other drugs in orally deliverable form, there remains a need for dosage forms of a therapeutic agent for treating sexual dysfunction in men and women, having one or more of the following benefits:

(a) low effective dosage rate;

(b) immediate absorption leading to rapid onset of therapeutic effect;

(c) minimal or no adverse side-effects;

(d) no requirement to be taken with water;

(e) reduced unpleasantness of taste;

(f) enhanced convenience of oral administration within a short interval prior to sexual activity; and

(g) discreet route of administration or method of use.

Other needs also remain in the art.

In one aspect, sexual dysfunction as addressed herein comprises sexual disorders including, without limitation, hypoactive sexual desire disorder, female sexual arousal disorder, male erectile disorder, female orgasmic disorder and male orgasmic disorder, all as defined in Diagnostic and Statistical Manual of Mental Disorders, 4th edition (DSM-IV) (1994), and DSM-IV Guidebook (1995), both published by American Psychiatric Press, Inc., Washington, D.C.

In another aspect, sexual dysfunction as addressed herein comprises diminishment of sexual desire, interest and/or function arising from primary diseases or conditions that are not sexual disorders in a strict sense. Such diseases and conditions include, without limitation, epilepsy, craniopharyngioma, hypogonadism and general psychiatric disorders such as depression. Sexual dysfunction as addressed herein additionally comprises sexual deficiencies following hysterectomy and/or oophorectomy as well as those arising as side effects of medication.

European Patent Application No. 0 960 621, incorporated herein by reference, discloses that sildenafil citrate has an unpleasant taste that cannot be completely masked by flavoring agents, and proposes rapidly disintegrating oral dosage forms of sildenafil in the form of its free base, which has extremely low solubility in water and is virtually tasteless.

International Patent Publication No. WO 99/66933, incorporated herein by reference, proposes intranasal administration of sildenafil, illustratively in the form of salts such as the hydrochloride salt, for treatment of erectile dysfunction. Dosage forms proposed include a nasal spray and an aqueous nasal gel. Aqueous solutions are said to be preferred. Rapid onset of therapeutic effect is contemplated; however, no solution is suggested to the problem of unpleasant taste arising from drainage of the drug into the mouth. Dosage rates are contemplated in WO 99/66933 to be lower than are required when the drug is orally administered; a 30 mg dose of sildenafil hydrochloride in the form of a nasal spray is exemplified. Also exemplified is a nasal spray formulation delivering 30 mg of sildenafil hydrochloride and 1 mg of apomorphine hydrochloride.

International Patent Publication No. WO 00/76509, incorporated herein by reference, also proposes nasal administration of apomorphine, illustratively as its hydrochloride salt.

European Patent Application No. 0 992 240, incorporated herein by reference, discloses cGMP-PDE inhibitory compounds said to be useful in treatment of male erectile dysfunction and proposes transmucomembranous administration, for example in the form of sublingual preparations, of such compounds.

Heaton (1996), “Buccal apomorphine”, Journal of Urology 155, 49, reports efficacy of a sublingual formulation of apomorphine in treatment of male non-organic erectile dysfunction.

U.S. Pat. No. 5,985,889 to El-Rashidy et al., incorporated herein by reference, proposes sublingual administration of apomorphine for treatment of male psychogenic erectile dysfunction. Various sublingual tablet formulations of apomorphine hydrochloride are disclosed therein.

International Patent Publication No. WO 00/35457, incorporated herein by reference, proposes use of apomorphine for treatment of male organic, e.g., vasculogenic, erectile dysfunction, and exemplifies use of a sublingual tablet formulation of apomorphine hydrochloride. WO 00/35457 further suggests that nausea, a common side effect of apomorphine, can be controlled by inclusion of an anti-emetic agent such as nicotine in the formulation.

U.S. Pat. No. 6,121,276 to El-Rashidy & Ronsen, incorporated herein by reference, discloses flavored sublingual tablets containing apomorphine hydrochloride and nicotine.

U.S. Pat. No. 5,994,363 to El-Rashidy & Ronsen, incorporated herein by reference, discloses a treatment regime with apomorphine that is said to reduce side effects such as nausea, vomiting, yawning and cardiovascular effects.

U.S. Pat. Nos. 5,624,677 and 5,888,534, both to El-Rashidy et al. and incorporated herein by reference, discloses a prolonged release sublingual formulation of apomorphine.

International Patent Publication No. WO 01/49292, incorporated herein by reference, discloses sublingual tablets of apomorphine providing prolonged release of the drug, said to be useful in treatment of Parkinson's disease.

International Patent Publication No. WO 00/42992, incorporated herein by reference, discloses a dosage unit comprising a water-soluble hydrocolloid and sildenafil citrate in a mucoadhesive film said to be suitable for application to the oral mucosa. Pharmacokinetic data presented in WO 00/42992 indicate no faster absorption into the bloodstream with sublingual application of such a film than with a commercial tablet formulation of sildenafil citrate (Viagra®) at the same dosage.

International Patent Publication No. WO 01/10406, incorporated herein by reference, discloses compositions said to be suitable for a wide range of routes of administration of sildenafil citrate, including buccal and sublingual routes. Preferred compositions disclosed are said to comprise a solution, gel, semisolid, suspension, metered dose device, transdermal patch or film.

International Patent Publication No. WO 02/05820, incorporated herein by reference, discloses film dosage forms comprising sildenafil citrate. These dosage forms are prepared by mixing a solid dispersion of sildenafil citrate and a water soluble sugar with a hydrocolloid and optionally other ingredients, and are said, upon placement on a mucosal surface, to form a coating that subsequently disintegrates and dissolves to release sildenafil.

### SUMMARY OF THE INVENTION

The present invention provides a rapid-onset pharmaceutical composition useful for treatment of sexual dysfunction, stimulation of sexual activity and enhancement of sexual desire, interest and performance in men and women. The composition is a dosage form comprising (a) a therapeutically or sexual-stimulatorily effective amount of a therapeutic agent having a molecular weight, excluding counterions, not greater than 250, and (b) at least one pharmaceutically acceptable excipient; and is adapted for delivery by a route of administration that entails interaction with the organs of taste yet has acceptable organoleptic properties.

In illustrative compositions the therapeutic agent is present in an amount of about 0.05 mg to about 10 mg per dose and comprises at least one compound of formula (I)

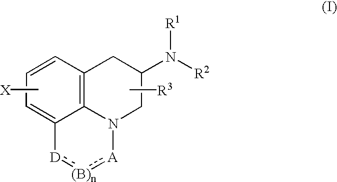

or a pharmaceutically acceptable salt thereof, wherein

R¹,R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl;

A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;

B is CH, CH₂, CHF, CHCl, CHBr, CHI, C═O, N, NH or NCH₃, and n is 0 or 1; and

D is CH, CH₂, CHF, CHCl, CHBr, CHI, C═O, O, N, NH or NCH₃; said compound of formula (I) or salt thereof being water-soluble.

A dosage form that interacts with the organs of taste in accordance with the present invention is described herein for convenience as “intraorally interacting” or “intraorally deliverable”. The route of administration of such a dosage form can illustratively be oral, buccal, sublingual, nasal or tracheal. “Intraorally interacting” dosage forms herein do not encompass solid dosage forms such as conventional tablets and capsules adapted for swallowing with water immediately on placement in the mouth.

Preferably a compound of formula (I) or salt thereof is selected having solubility in water at 20-25° C. of at least about 10 g/l.

Preferred compounds useful in compositions of the invention are those disclosed generically or specifically in above-cited U.S. Pat. No. 5,273,975. Especially preferred compounds are those of formula (II)

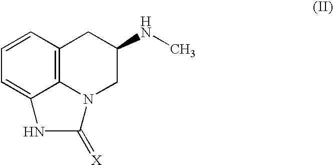

wherein X is O or S, and pharmaceutically acceptable salts thereof.

A “therapeutically effective amount” herein is an amount sufficient to improve sexual desire, interest or performance in a subject having a sexual dysfunction condition. A “sexual-stimulatorily effective amount” herein is an amount sufficient to improve sexual desire, interest or performance in a subject whether or not the subject has a sexual dysfunction condition. It is preferred that the amount of the compound of formula (I) or salt thereof be lower than an amount causing significant side-effects; in general it will be found that dosage amounts lower than about 5 mg, especially lower than about 3 mg, are relatively free of such side-effects.

Dosage forms suitable for intraoral delivery according to the present invention include without restriction

(a) buccal and sublingual tablets including those permitting absorption of the therapeutic agent through the oral mucosa;

(b) mucoadhesive films;

(c) oral strips;

(d) chewable tablets;

(e) rapidly disintegrating oral dosage forms such as fast-melt tablets;

(f) lozenges and pastilles including those permitting oropharyngeal absorption of the therapeutic agent;

(g) breath-fresheners such as breath-mints;

(h) chewing gums and chewing candy;

(i) lollipops and popsicles;

(j) food adjuncts, e.g., broths, bouillon cubes and granules, puddings, jellies, spreads, etc.;

(k) candies and chocolates;

(l) periodontal gels;

(m) mouthwashes;

(n) oral and nasal drops and sprays;

(o) dosage forms adapted for inhalation as an aerosol or vapor;

(p) elixirs, solutions, suspensions and other orally administered liquid dosage forms;

(q) powders, granules and tablets for dissolution or dispersion in water prior to oral administration; and

(r) effervescent tablets and granules.

All of the above dosage forms interact in normal use with the organs of taste, unlike conventional tablets and capsules that are normally swallowed with water and are not retained long enough in the mouth to so interact. A particularly useful intraorally deliverable dosage form of the present invention is a rapidly disintegrating oral formulation that dissolves in the mouth without need for drinking water or other fluid. Such a formulation is described herein as a “fast-melt” formulation. Other particularly useful intraorally deliverable dosage forms of the present invention are a breath-mint, a sublingual tablet, a chewing gum, a mucoadhesive film and an oral strip.

A dosage form having “acceptable organoleptic properties” herein is one that, upon intraoral interaction in an amount providing a single dose of the therapeutic agent, does not have an excessively unpleasant taste, smell or mouth feel, for example a pronouncedly bitter taste, as perceived by a majority of subjects. Acceptable organoleptic properties can arise, for example, where the therapeutic agent is selected to have acceptable taste; where the dose is so low that any unpleasant taste of the therapeutic agent is greatly diminished; where an unpleasant taste of the therapeutic agent is masked or balanced by one or more flavoring agents, sweeteners, or other excipients; or where an unpleasant-tasting therapeutic agent is encapsulated to reduce direct contact between the therapeutic agent and the organs of taste.

Contemplated therapeutic agents, in particular compounds of formula (II) and salts thereof, when formulated in dosage forms as described herein, can be effective at surprisingly low doses. At such low doses, despite the high aqueous solubility of compounds of formula (II) and in particular of their salts, there is generally no pronounced taste associated with the therapeutic agent. Even if a taste is detectable, it is relatively easily masked or balanced by excipients and encapsulation is normally not required.

Especially preferred dosage forms of the invention are adapted for discreet self-administration. By “discreet self-administration” herein is meant self-administration shortly prior to sexual activity in a way that does not draw attention of a sexual partner to, or emphasize, the existence of a sexual dysfunction, a need for therapy or a need or desire for enhancement of sexual performance. The combination of discreetness and rapid onset that is permitted by the present invention provides a benefit in spontaneity; by contrast, prior art compositions for treating sexual dysfunction can be seriously compromised in their effectiveness if their self-administration requires premeditation and/or cannot be done discreetly, such self-administration being thereby not conducive to spontaneity. In particular, the present invention does not involve self-injection, and generally does not require water or other drink as an aid to swallowing.

Also provided by the present invention are methods of use of compositions of the present invention for treatment of sexual dysfunction and for enhancement of sexual desire, interest or performance, and a method of use of a composition of the invention for preparing a medicament. Other features of this invention will be in part apparent and in part pointed out hereinafter.

### BRIEF DESCRIPTION OF THE DRAWING

FIG. 1 is a graphical representation of the relationship between the molecular weight of a drug in its “free” form as defined herein (MW_{free}) and Tₘₐₓ following sublingual administration.

### DETAILED DESCRIPTION OF THE INVENTION

As indicated above, the present invention provides, in one embodiment, a pharmaceutical dosage form suitable for oral administration to a human subject, the dosage form comprising pharmaceutically acceptable excipients having mixed therewith at least one agent, effective in treatment of sexual dysfunction, having a molecular weight, excluding counterions, not greater than 250, in a therapeutically or sexual-stimulatorily effective total amount; wherein the dosage form is adapted for intraoral delivery as defined herein and has acceptable organoleptic properties.

The invention arises in part from a surprising result arrived at by a new analysis of published data on pharmacokinetics in human subjects of twelve different drugs administered sublingually. It is noted that with conventional oral administration (followed immediately by swallowing of the dosage form), absorption of a drug occurs predominantly or entirely in the gastrointestinal tract, whereas with sublingual administration there is opportunity for the drug to be absorbed predominantly or entirely via the oral mucosa.

The new analysis focuses on Tₘₐₓ which is a pharmacokinetic parameter, conventionally computed from human pharmacokinetic data, expressing the time required following administration for blood serum concentration of an administered drug to reach a maximum value.

The analysis relates sublingual Tₘₐₓ (Tₘₐₓ following sublingual administration) to molecular weight of the drug in its “free” form, i.e., excluding counterions in the case of drugs that are salts, a parameter herein abbreviated as “MW_{free}” or “free molecular weight”. A close and hitherto undisclosed correlation between MW_{free} and sublingual Tₘₐₓ is revealed by this analysis. More specifically, the three drugs in this analysis having free molecular weight lower than 250 form a cluster having on average a much shorter Tₘₐₓ (13 minutes) than the nine drugs having free molecular weight higher than 250 (on average, 69 minutes).

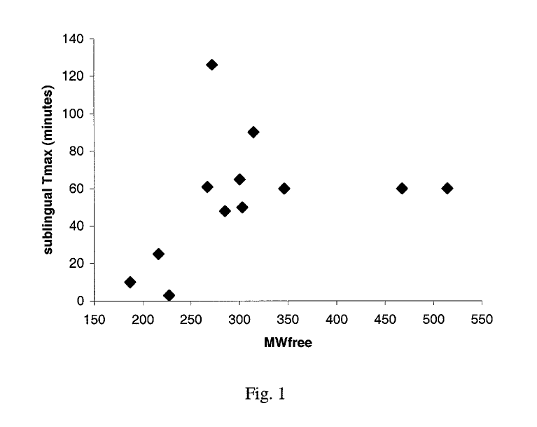

The data used in the analysis are as shown in Table 1 below and the relationship between MW_{free} and sublingual Tₘₐₓ is shown graphically in FIG. 1. Tₘₐₓ data are derived from the publications listed below. Except where noted below, Tₘₐₓ data relate to drug administered as free base.

Selegiline: Mahmood (1997), Clinical Pharmacokinetics 33(2), 91-102.

Methoxsalen: Shephard et al. (2001), Photodermatology, Photoimmunology & Photomedicine 17, 11-21.

Nitroglycerin: http://www.tomescps.com/hcsdata/de/de0035.htm.

Apomorphine: http://www.tomescps.com/hcsdata/de/de1066.htm.

Sotalol: Deneer et al. (1998), British Journal of Clinical Pharmacology 45(5), 485-490.

Morphine: Taylor (1996), Journal of Pharmacy and Pharmacology 48(12).

Temazepam: Russell & Babcock (1988), European Journal of Clinical Pharmacology 35(4).

Scopolamine: http://jpet.aspetjournals.org/cgi/content/full/296/1/121#sec3.

Clomipramine: Dong Yoo et al. (1999), Journal of Pharmaceutical Sciences 88(11).

Nifedipine: http://www.tomescps.com/hcsdata/de/de0598.htm.

Buprenorphine: Drug and Alcohol Dependence (1999) 56(1), 55-60.

Sildenafil: above-cited International Patent Publication No. WO 00/42992.

TABLE 1
Relationship of sublingual Tₘₐₓ to MW_{free}

| Drug | MW_{free} | sublingual Tₘₐₓ (minutes) |
|---|---|---|
| selegiline | 187.1 | 10 |
| methoxsalen | 216.2 | 25 |
| nitroglycerin | 227.1 | 3 |
| apomorphine^{1,2} | 267.3 | 61 |
| sotalol | 272.4 | 126 |
| morphine³ | 285.3 | 48 |
| temazepam | 300.7 | 65 |
| scopolamine⁴ | 303.4 | 50 |
| clomipramine² | 314.9 | 90 |
| nifedipine | 346.6 | 60 |
| buprenorphine² | 467.7 | 60 |
| sildenafil^{1,5} | 514.7 | 60 |

¹drug known to be therapeutically effective in treatment of sexual dysfunction
²hydrochloride salt
³sulfate salt
⁴hydrobromide salt
⁵citrate salt

Accordingly, it is contemplated that a drug having a free molecular weight no greater than 250, more particularly no greater than 235, most particularly no greater than 220, for example from about 150 to about 220, said drug being therapeutically effective in treatment of sexual dysfunction, has especially useful properties of rapid absorption and consequently fast onset of therapeutic or sexual-stimulatory effect when delivered intraorally, for example sublingually.

In another embodiment, the present invention provides a pharmaceutical dosage form suitable for oral administration to a human subject, the dosage form comprising pharmaceutically acceptable excipients having mixed therewith an agent comprising at least one water-soluble compound of formula (I) or pharmaceutically acceptable water-soluble salt of a compound of formula (I) in a therapeutically or sexual-stimulatorily effective total amount; wherein the dosage form is adapted for intraoral delivery as defined herein and has acceptable organoleptic properties.

According to either of the above embodiments, it is preferred that the agent be therapeutically or sexual-stimulatorily effective in an amount of about 0.05 mg to about 10 mg per dose.

The invention is described herein with particular reference to compounds of formula (II) and their salts, more particularly sumanirole, (R)-5,6-dihydro-5-(methylamino)-4H-imidazo[4,5-ij]-quinolin-2(1H)-one (free molecular weight 203), and its salts; and (R)-5,6-dihydro-5-(methylamino)-4H-imidazo[4,5-ij]-quinoline-2(1H)-thione (free molecular weight 219) and its salts. However, it is to be understood that sumanirole or (R)-5,6-dihydro-5-(methylamino)-4H-imidazo[4,5-ij]-quinoline-2(1H)-thione can be replaced in part or in whole in a composition of the invention with another compound of formula (I) or a salt thereof, particularly a compound as disclosed in above-cited U.S. Pat. No. 5,273,975 or a salt thereof.

Pharmaceutically acceptable salts of a compound of formula (II) include without restriction salts of the following acids: hydrochloric, hydrobromic, sulfuric, methanesulfonic, phosphoric, nitric, benzoic, citric, tartaric, fumaric and maleic acids, and mono- and dicarboxylic acids of formula CH₃—(CH₂)ₙ—COOH and HOOC—(CH₂)ₙ—COOH where n is 0 to 4, for example malonic acid.

Particularly preferred salts are the hydrochloride salt and the maleate, i.e., (Z)-2-butenedioate, salt.

Compounds of formula (II) and their salts can be prepared by processes known per se, including processes described in patent literature cited herein. However, the present invention is not restricted by the process used to prepare the therapeutic agent.

The terms “therapeutically effective” and “sexual-stimulatorily effective” are defined hereinabove, in relation to the amount of a compound of formula (II) or salt thereof present in a dosage form of the invention, as effective to improve sexual desire, interest or performance. Such desire, interest or performance can relate to any sexual activity but in particular relates to sexual intercourse whether or not accompanied by orgasm, ejaculation, masturbation and/or sexual foreplay.

A sumanirole dosage form of the invention preferably contains about 0.05 to about 5 mg, more preferably about 0.1 to about 5 mg, still more preferably about 0.2 to about 5 mg, even more preferably about 0.5 to about 5 mg, of sumanirole free base equivalent, as free base or as salt. In one embodiment the dosage form contains about 0.25 to about 3 mg, for example about 1 to about 3 mg, of sumanirole free base equivalent, as free base or as salt. If desired, the sumanirole can be only partially neutralized with acid so that free base coexists with salt in the dosage form.

A dosage form of the invention wherein the therapeutic agent is (R)-5,6-dihydro-5-(methylamino)-4H-imidazo[4,5-ij]-quinoline-2(1H)-thione preferably contains about 0.05 to about 5 mg, more preferably about 0.1 to about 3 mg, and most preferably about 0.25 to about 2 mg, of (R)-5,6-dihydro-5-(methylamino)-4H-imidazo[4,5-ij]-quinoline-2(1H)-thione free base equivalent, as free base or as salt. In one embodiment the dosage form contains about 0.1 to about 3 mg, for example about 0.25 to about 1 mg, of (R)-5,6-dihydro-5-(methylamino)-4H-imidazo[4,5-ij]-quinoline-2(1H)-thione free base equivalent, as free base or as salt. If desired, the (R)-5,6-dihydro-5-(methylamino)-4H-imidazo[4,5-ij]-quinoline-2(1H)-thione can be only partially neutralized with acid so that free base coexists with salt in the dosage form.

In one embodiment the compound of formula (I) is present in a dosage form of the invention in a therapeutically or sexual-stimulatorily effective amount of less than 1 mg, for example about 0.05 mg to about 0.75 mg. Surprisingly a dosage form of the invention having such a low amount of the active agent can exhibit a desired degree of efficacy; further, any unpleasant taste resulting from intraoral interaction by the dosage form is minimized or absent.

As indicated above, the dosage form is adapted for delivery by a route of administration that entails intraoral interaction. In one embodiment, the dosage form is adapted for a nasal route of administration, and absorption of the drug occurs at least in part from the nasal cavity. In another embodiment, the dosage form is adapted for a tracheal route of administration, and absorption of the drug occurs at least in part in the respiratory tract. In yet another embodiment, the dosage form is adapted for an oral, including buccal or sublingual, route of administration, and absorption of the drug occurs in the oral cavity and/or in the gastrointestinal tract.

A liquid formulation suitable for instilling or spraying into the nasal cavity can be prepared by any suitable process disclosed in the art for such formulations, illustratively including processes disclosed in the publications individually listed below, with modification as required for a therapeutic agent that is sumanirole or another compound of formula (I), or a salt thereof. Such modification will readily be made by one of skill in the art of pharmaceutical formulation.

Above-cited International Patent Publication No. WO 99/66933.

Above-cited International Patent Publication No. WO 00/76509.

An illustrative liquid formulation of the invention, suitable for nasal administration but also for oral administration, comprises a solution of a salt, e.g., the maleate salt, of sumanirole or (R)-5,6-dihydro-5-(methylamino)-4H-imidazo[4,5-ij]-quinoline-2(1H)-thione (the “active agent”) in an aqueous medium, and has the following composition:

| | |
|---|---|
| active agent | 1 mg/ml free base equivalent |
| sodium benzoate (preservative) | 0.125% |
| 10% hydrochloric acid solution | 26.5% |
| 10% sodium hydroxide solution | 20.5% |
| water for injection | q.s. to 100% |

The hydrochloric acid and sodium hydroxide are present to adjust pH to a desired range. Optionally other ingredients, including viscosity modifiers, sweetening agents and agents to enhance flavor and/or aroma, can be added, with adjustment where necessary of the selection and amount of preservative.

Illustratively, where the active agent is (R)-5,6-dihydro-5-(methylamino)-4H-imidazo[4,5-ij]-quinoline-2(1H)-thione maleate, a suitable dosage amount of the active agent is about 0.1 mg to about 1 mg per dose. Nasal administration of the 1 mg/ml composition described above by means of a spray package, e.g., an aerosol dispenser, that dispenses, illustratively, 0.125 ml per spray will deliver 0.125 mg of active agent per spray. Selection of active agent loading and spray dispenser is preferably made such that about one to about four sprays delivers a therapeutic dose. Administration can be made to one or to both nostrils.

Even where the 1 mg/ml composition described above is administered orally rather than nasally, no unacceptable taste is reported.

A nasal composition, or alternatively an oral spray or aerosol formulation of the invention suitable for absorption by the mucosa of the intraoral cavity, can optionally further comprise an oral absorption enhancer such as a cyclodextrin (e.g., hydroxypropyl-β-cyclodextrin) or an orally acceptable surfactant, as disclosed in International Patent Publication No. WO 00/47203, incorporated herein by reference.

Preferred oral dosage forms of the invention include fast-melt formulations, breath-freshening pastilles, chewing gums, sublingual tablets, mucoadhesive films and oral strips, all of which are well adapted for discreet self-administration.

Preferred therapeutic agents useful in the present invention are water-soluble, and typically dissolve in saliva concurrently with dissolution of the carrier in the oral cavity. The agent can be absorbed at least in part by the oral mucosa and at least in part in the gastrointestinal tract after swallowing. It is not a requirement of the present invention that there be a significant element of buccal or oral absorption, but it is believed, without being bound by theory, that if such absorption occurs to a substantial degree the onset of therapeutic effect is more rapid. Even if there is no substantial buccal or oral absorption, however, rapid onset of therapeutic effect can still occur. It is believed, again without being bound by theory, that this rapid onset is permitted by the fact that the agent is already in solution when swallowed.

Breath-freshening pastilles have not hitherto been widely adopted in the pharmaceutical formulation art. These are usually very small articles having a sweet carrier and a breath-freshening flavoring agent, typically peppermint or spearmint, but optionally a non-mint flavoring agent such as wintergreen or cinnamon. Familiar brands of breath-mint confectionery include Tic Tac® mints of Ferrero. According to the present invention a breath-mint, or analogous article having a flavoring other than mint, can be used as a dosage form for intraoral delivery of any agent effective in treatment of sexual dysfunction contemplated herein.

Preferred breath-freshening pastille formulations of the invention have an inner core enveloped in a hard sugar-based coating. Upon placement in the mouth, the coating, which typically has a sweet taste, rapidly dissolves to permit release of flavoring agents from the core. It is generally preferred that the agent be incorporated in the coating, to facilitate early release of the agent and thereby rapid onset of therapeutic effect. Optionally, however, the agent can be present in both the coating and the core, or only in the core.

Breath-mints can be prepared by any process known in the art, including those involving one or more of direct compression, extrusion/spheronization, co-extrusion (e.g. twisted ribboning), conventional pan-coating and fluidized bed coating. Illustratively, suitable processes are described in the publications individually listed below and incorporated herein by reference, with modification as required for incorporation of a suitable agent effective in treatment of sexual dysfunction such as a compound of formula (I), or a salt thereof.

U.S. Pat. No. 4,847,090 to Della Posta & Piano.

U.S. Pat. No. 5,431,918 to Damonte & Ferrero.

U.S. Pat. No. 6,083,527 to Thistle.

European Patent Application No. 0 940 084.

Breath-mints are composed primarily of a sweet-tasting sugar, e.g., sucrose or sorbitol, and can contain minor amounts of other ingredients such as dextrin, starch, gum arabic, carnauba wax, oils, magnesium stearate, magnesium oxide, magnesium carbonate, povidone, polyethylene glycol (PEG), propylene glycol, citric acid, and natural and/or artificial coloring and/or flavoring agents. Any tableting process can be used in manufacture of breath-mints, but preferably a conventional pan-coating process is used.

An illustrative breath-mint formulation of the invention containing as active agent a salt, e.g., the maleate salt, of sumanirole or (R)-5,6-dihydro-5-(methylamino)-4H-imidazo[4,5-ij]-quinoline-2(1H)-thione has the following composition:

| active agent | 0.1-3% free base equivalent |
|---|---|
| sugar | 70-99% |
| non-sugar carrier (e.g., dextrin) | 0-20% |
| oil (e.g., corn oil) | 0-0.5% |
| plasticizer (e.g., PEG) | 0-0.8% |
| flavoring agent | 0.2-3% |

all percentages being by weight.

Preferred chewing gum formulations of the invention have an inner core of chewing gum enveloped in a hard sugar-based coating. Chewing gums that have typically been used as dosage forms for delivery of a drug, for example nicotine or lobeline in smoking-cessation chewing gums, have the drug incorporated in the chewing gum matrix; however, according to the present invention it is preferred that the agent effective in treatment of sexual dysfunction be incorporated in the coating, as in the preferred breath-freshening pastille formulations described above.

For purposes of illustration, a chewing gum of the invention can be prepared by appropriate modification of a process described for a nicotine chewing gum in U.S. Pat. No. 3,901,248 to Ferno et al., which is incorporated herein by reference. The chewing gum base can be of natural origin, e.g., chicle, jelutong, lechi di caspi, soh, siak, katiau, sorwa, balata, pendare, perillo, malaya and percha gums, caoutchouc, natural resins, etc., or synthetic, e.g., polyvinylacetate, Dreyco™ commercial gum base, polyvinyl esters, polyisobutylene and non-toxic butadienestyrene lattices. Plasticizers are normally incorporated into the chewing gum base to provide a desirable viscosity, consistency and texture for chewing. Plasticizers typically also act as moisture-retaining agents. Suitable plasticizers include lecithin, lanolin, hydrogenated coconut oil, hydrogenated cottonseed oil, mineral oil, olive oil, vaseline, carnauba wax, candelilla wax, paraffin, beeswax, stearic acid, glyceryl monostearate, glycerin, honey, propylene glycol, hexylene glycol, sorbitol, etc. Sugars such as sucrose, sorbitol and glucose (e.g., in the form of corn syrup) are normally the most abundant ingredient. Other ingredients optionally present include cerelose, mannitol, diastatic malt, starch, calcium carbonate, talc, defatted cocoa, coloring and flavoring agents.

An illustrative chewing gum formulation of the invention containing as active agent a salt, e.g., the maleate salt, of sumanirole or (R)-5,6-dihydro-5-(methylamino)-4H-imidazo[4,5-ij]-quinoline-2(1H)-thione has the following composition:

| active agent | 0.1-3% free base equivalent |
|---|---|
| citric acid, anhydrous | 0-9% |
| sodium bicarbonate | 0-15% |
| natural gum base | 60-95% |
| pregelatinized starch | 2-9% |
| flavoring agent | 0.5-2% |
| coloring agent | 0-0.3% |
| talc | 0-1.5% |
| magnesium stearate | 0.5-1.5% |

all percentages being by weight.

Another illustrative chewing gum formulation of the invention containing as active agent a salt, e.g., the maleate salt, of sumanirole or (R)-5,6-dihydro-5-(methylamino)-4H-imidazo[4,5-ij]-quinoline-2(1H)-thione comprises a core having the following composition:

| | |
|---|---|
| natural gum base | 20% |
| powdered sorbitol | 59% |
| corn syrup | 20% |
| glycerin | 0.5% |
| flavoring agent | 0.5% |

all percentages being by weight, and a coating having the following composition:

| | |
|---|---|
| active agent | 0.1-3 mg per piece |
| coating sugar | q.s. |

Pieces can be of any convenient size, but illustratively have a total weight of about 2 g.

Incorporation of the active agent in a sugar coating of a breath-mint, chewing gum or other dosage form can be achieved by including in the coating formula an aqueous solution of the active agent at a concentration calculated to provide the desired dose per piece.

Where the dosage form is a modified confectionery item, such as a breath-mint or a chewing gum, it is preferred that the individual pieces have distinctive design features (e.g., shape, color, surface marking or embossing, etc.) to avoid possible confusion with confectionery.

Sublingual formulations of the invention can be prepared by any suitable process known in the art, with modification as appropriate to include an agent effective in treatment of sexual dysfunction.

An illustrative sublingual tablet of the invention containing as active agent a salt, e.g., the maleate salt, of sumanirole or (R)-5,6-dihydro-5-(methylamino)-4H-imidazo[4,5-ij]-quinoline-2(1H)-thione has the following composition:

| active agent | 0.1-3% free base equivalent |
|---|---|
| mannitol | 50-90% |
| powdered sorbitol | 10-40% |
| hydroxypropylcellulose | 0-10% |
| xanthan gum | 0-5% |
| flavoring agent | 0-0.5% |
| coloring agent | 0-0.5% |
| colloidal silicon dioxide | 0-1% |
| magnesium stearate | 0.5-5% |

all percentages being by weight.

Another illustrative sublingual tablet of the invention containing as active agent a salt, e.g., the maleate salt, of sumanirole or (R)-5,6-dihydro-5-(methylamino)-4H-imidazo[4,5-ij]-quinoline-2(1H)-thione has the following composition:

| active agent | 0.1-3% free base equivalent |
|---|---|
| lactose monohydrate | 50-85% |
| pregelatinized starch | 10-45% |
| xanthan gum | 0-5% |
| flavoring agent | 0-0.5% |
| coloring agent | 0-0.5% |
| colloidal silicon dioxide | 0-1% |
| magnesium stearate | 0.5-5% |

all percentages being by weight.

Yet another illustrative sublingual tablet of the invention containing as active agent a salt, e.g., the maleate salt, of sumanirole or (R)-5,6-dihydro-5-(methylamino)-4H-imidazo[4,5-ij]-quinoline-2(1H)-thione has the following composition:

| active agent | 0.1-3% free base equivalent |
|---|---|
| microcrystalline cellulose | 30-70% |
| pregelatinized starch | 25-65% |
| croscarmellose sodium | 0-10% |
| xanthan gum | 0-5% |
| flavoring agent | 0-0.5% |
| coloring agent | 0-0.5% |
| colloidal silicon dioxide | 0-1% |
| magnesium stearate | 0.5-5% |

all percentages being by weight.

Sublingual tablets such as those illustratively described above can be uncoated, or optionally can be coated. Any known coating material can be used, typically to provide a weight gain of about 0.1% to about 3%. It has been found that a particularly desirable coating material, not previously described for sublingual tablets, is one based on gellan gum, alone or in combination with other polymers. A gellan gum coating can enhance “seating” of a tablet in the sublingual space, thereby improving contact with the mucosa. In addition, the gellan gum coating can provide a degree of mucoadhesion. A gellan gum coating is especially useful where the tablet has low hardness (a feature desirable for rapid disintegration in the mouth), more traditional coatings being difficult to apply to such a tablet without excessive breakage and attrition of the tablet.

A mucoadhesive film formulation of the invention, suitable for example for sublingual or buccal application, can be prepared substantially as disclosed in above-cited International Patent Publication No. WO 00/42992, with modification as required for a therapeutic agent as contemplated herein.

An oral strip formulation of the invention can, for example, resemble the Cool Mint Listerine PocketPaks™ product of Pfizer Inc., as disclosed for example at http://www.prodhelp.com/oral_care17.shtml, except for incorporation in the formulation of a therapeutic agent as contemplated herein. A preferred oral strip of the invention is a very thin starch-based film having impregnated therein a therapeutically or sexual-stimulatorily effective amount of a compound of formula (II) or salt thereof.

Fast-melt formulations are well known in the pharmaceutical formulation art, and exhibit rapid disintegration, usually associated with one or more carrier excipients, typically sugars, and concomitant rapid dissolution or dispersion of the therapeutic agent in the oral cavity, usually without need for water other than that contained in saliva. The term “oral cavity” includes the entire interior of the mouth, including not only the buccal cavity (that part of the oral cavity anterior to the teeth and gums) but also the sublingual and supralingual spaces.

Processes suitable for preparing a fast-melt formulation of the invention include, without limitation, processes substantially as disclosed in any of the patents listed below, with modification as required for a therapeutic agent as contemplated herein. Such modification will readily be made by one of skill in the art of pharmaceutical formulation. These patents are incorporated herein by reference.

U.S. Pat. No. 3,885,026 to Heinemann & Rothe.

U.S. Pat. No. 4,134,943 to Knitsch et al.

U.S. Pat. No. 4,305,502 to Gregory & Ho.

U.S. Pat. No. 4,371,516 to Gregory et al.

U.S. Pat. No. 4,414,198 to Michaelson.

U.S. Pat. No. 4,855,326 to Fuisz.

U.S. Pat. No. 4,946,684 to Blank et al.

U.S. Pat. No. 5,073,374 to McCarty.

U.S. Pat. No. 5,178,878 to Wehling et al.

U.S. Pat. No. 5,298,261 to Pebley et al.

U.S. Pat. No. 5,401,514 to Juch et al.

U.S. Pat. No. 5,417,985 to Coutel et al.

U.S. Pat. No. 5,464,632 to Cousin et al.

U.S. Pat. No. 5,466,464 to Masaki & Ban.

U.S. Pat. No. 5,082,667 to Van Scoik.

U.S. Pat. No. 5,501,861 to Makino et al.

U.S. Pat. No. 5,503,846 to Wehling et al.

U.S. Pat. No. 5,518,730 to Fuisz.

U.S. Pat. No. 5,576,014 to Mizumoto et al.

U.S. Pat. No. 5,587,172 to Cherukuri et al.

U.S. Pat. No. 5,587,180 to Allen & Wang.

U.S. Pat. No. 5,607,697 to Alkire et al.

U.S. Pat. No. 5,622,719 to Myers et al.

U.S. Pat. No. 5,653,926 to Bogue & Myers.

U.S. Pat. No. 5,662,849 to Bogue & Myers.

U.S. Pat. No. 5,733,577 to Myers et al.

U.S. Pat. No. 5,762,961 to Roser & Blair.

U.S. Pat. No. 5,807,576 to Allen et al.

U.S. Pat. No. 5,837,285 to Nakamichi et al.

U.S. Pat. No. 5,869,098 to Misra et al.

U.S. Pat. No. 5,876,759 to Gowan.

U.S. Pat. No. 5,939,091 to Eoga & Valia.

U.S. Pat. No. 5,958,453 to Ohno et al.

U.S. Pat. No. 6,010,719 to Remon & Corveleyn.

U.S. Pat. No. 6,024,981 to Khankari et al.

Some of the above, and other, approaches to formulating fast-melt tablets have been summarized by Chang et al. in Pharmaceutical Technology, Jun. 2000, pp. 52-58.

Each of these processes involves a step of preparing a moldable blend comprising the therapeutic agent, e.g., a compound of formula (II), and an excipient carrier system, and a step of shaping the moldable blend to form a solid dosage form such as a tablet, lozenge or wafer. Typically the excipient carrier system consists predominantly of one or more carbohydrates; that is to say that carbohydrates constitute more than 50% by weight of all excipients in the moldable blend formed with the therapeutic agent.

The moldable blend can be liquid or semi-liquid, as for example a paste, in which case the shaping step can be accomplished by placement in a suitable mold and drying, for example by heat, by vacuum or by freeze-drying. Alternatively the shaping step can be accomplished by compression, for example in a tableting press. The process can optionally include a step of extrusion prior to tableting.

Excipient ingredients forming the carrier system for the therapeutic agent in a formulation of the invention include at least one pharmaceutically acceptable carbohydrate. The carbohydrate(s) can function as bulking agents, as swelling agents, as wicking agents, as binders and/or in other ways. Illustratively, the carbohydrate(s) can be selected from natural and modified celluloses, e.g., microcrystalline cellulose, hydroxypropylmethylcellulose, methylcellulose, ethylcellulose, hydroxypropylcellulose, carboxymethylcellulose, sodium carboxymethylcellulose, etc., natural and modified starches, e.g., corn starch, pregelatinized starch, sodium starch glycolate, etc., and mono-, di- and oligosaccharides having up to 6 saccharide units, including sugars and sugar alcohols, e.g., erythritol, glucose, lactose, maltitol, maltose, mannitol, sorbitol, sucrose, xylitol, etc.

The term “saccharide” is used herein to denote a sugar or sugar alcohol having 1 to about 6 saccharide units. It is preferred that at least one carbohydrate substantially present in the carrier system is selected from sugars and sugar alcohols, more preferably those exhibiting rapid dissolution in the mouth, most preferably those exhibiting such rapid dissolution and providing a sweet taste. Sugars and sugar alcohols having high moldability, e.g., maltitol, maltose and sorbitol, as well as sugars and sugar alcohols having low moldability, particularly when in finely particulate as opposed to granular form, e.g., glucose, lactose, mannitol, sucrose and xylitol, can be useful. Mono- and disaccharides are generally preferred.

Selection of suitable carbohydrates can readily be made by reference to the above-cited patents describing processes for preparing fast-melt pharmaceutical formulations.

One or more carbohydrates are typically present in a fast-melt formulation of the invention in a total amount of about 20% to about 95% by weight of the formulation.

Optionally, a fast-melt formulation of the invention can contain one or more additional pharmaceutically acceptable excipients including, but not limited to, wetting agents, water-soluble lubricants, water-insoluble lubricants, disintegrants, glidants, sweeteners, flavoring agents, effervescent agents, colorants, etc. Such optional additional components should be physically and chemically compatible with the other ingredients of the molded article and must not be deleterious to the recipient. Selection of suitable excipients can readily be made by reference to the above-cited patents describing processes for preparing fast-melt pharmaceutical formulations. Some of the excipients mentioned illustratively below are carbohydrates and are therefore already included in the category of carbohydrates described above.

Pharmaceutically acceptable wetting agents that can optionally be present in a fast-melt formulation of the invention include, individually or in combination, surfactants, hydrophilic polymers and certain clays. Non-limiting examples of surfactants that can be useful include quaternary ammonium compounds, e.g., benzalkonium chloride, benzethonium chloride and cetylpyridinium chloride, dioctyl sodium sulfosuccinate, polyoxyethylene alkylphenyl ethers, e.g., nonoxynol 9, nonoxynol 10, and octoxynol 9, poloxamers (polyoxyethylene and polyoxypropylene block copolymers), polyoxyethylene fatty acid glycerides and oils, e.g., polyoxyethylene (8) caprylic/capric mono- and diglycerides, polyoxyethylene (35) castor oil and polyoxyethylene (40) hydrogenated castor oil, polyoxyethylene alkyl ethers, e.g., polyoxyethylene (20) cetostearyl ether, polyoxyethylene fatty acid esters, e.g., polyoxyethylene (40) stearate, polyoxyethylene sorbitan esters, e.g., polysorbate 20 and polysorbate 80, propylene glycol fatty acid esters, e.g., propylene glycol laurate, sodium lauryl sulfate, fatty acids and salts thereof, e.g., oleic acid, sodium oleate and triethanolamine oleate, glyceryl fatty acid esters, e.g., glyceryl monostearate, sorbitan esters, e.g., sorbitan monolaurate, sorbitan monooleate, sorbitan monopalmitate and sorbitan monostearate, tyloxapol, and mixtures thereof. Sodium lauryl sulfate is a preferred wetting agent in formulations of the invention.

One or more wetting agents, if desired, are present in a formulation of the invention in a total amount of about 0.05% to about 5% by weight of the formulation.

Pharmaceutically acceptable water-insoluble lubricants that can optionally be present in a fast-melt formulation of the invention include glyceryl behapate, stearates (e.g., magnesium, calcium and sodium stearates), stearic acid, hydrogenated vegetable oils, colloidal silica, talc, waxes and mixtures thereof. Optionally a water-insoluble lubricant can be used in mixture with a wetting agent, as for example in calcium stearate/sodium lauryl sulfate mixtures (e.g., Sterowet™). Magnesium stearate, stearic acid and mixtures thereof are preferred water-insoluble lubricants.

One or more water-insoluble lubricants, if desired, are present in a formulation of the invention in a total amount of about 0.05% to about 5% by weight of the formulation.

Pharmaceutically acceptable water-soluble lubricants that can optionally be present in a formulation of the invention include boric acid, sodium benzoate, sodium acetate, sodium fumarate, sodium chloride, DL-leucine, polyethylene glycols (e.g., Carbowax™ 4000 and Carbowax™ 6000), sodium oleate and mixtures thereof.

One or more water-soluble lubricants, if desired, are present in a formulation of the invention in a total amount of about 0.05% to about 5% by weight of the formulation.

Pharmaceutically acceptable disintegrants that can optionally be present in a formulation of the invention include starches, sodium starch glycolate, clays, e.g., Veegum™ HV, celluloses, e.g., purified cellulose, methylcellulose, sodium carboxymethylcellulose, carboxymethylcellulose, etc., croscarmellose sodium, alginates, pregelatinized corn starches, e.g., National™ 1551 and National™ 1550, crospovidone, gums, e.g., agar, guar, locust bean, karaya, pectin and tragacanth gums, and mixtures thereof. Croscarmellose sodium and sodium starch glycolate are preferred disintegrants.

One or more disintegrants, if desired, are present in a formulation of the invention in a total amount of about 0.5% to about 7.5% by weight of the formulation.

Optionally, an effervescent salt can be used as a disintegrant and to enhance organoleptic properties of a fast-melt formulation of the invention.

Pharmaceutically acceptable glidants that can optionally be present in a formulation of the invention, for example to enhance flow of tableting material into tablet dies, to prevent sticking of tableting material to punches and dies, or to produce tablets having a sheen, include silicon dioxide products such as fumed silica (e.g., Cab-O-Sil™ of Cabot Corp. and Aerosil™ of Degussa).

Pharmaceutically acceptable sweeteners that can optionally be present in a molded article of the invention in a sweetening effective amount include sucrose, mannitol, propylene glycol, sodium saccharin, acesulfame K, neotame, aspartame, etc.

Pharmaceutically acceptable flavoring agents that can optionally be present in a molded article of the invention in a flavoring effective amount include peppermint, spearmint, cinnamon, grape, cherry, strawberry, lemon, etc.

Optionally, a solid fast-melt formulation can be scored or otherwise provided with means for convenient breaking into unit-dose segments, but is preferably a self-contained dosage form delivering a single unit dose. In a preferred embodiment, the formulation is an oral fast-melt tablet.

Tablets of the invention can be made to any desired size, for example 8 mm, 10 mm, 12 mm, etc., shape, for example round, oval, oblong, etc., weight, and thickness. Optionally, tablets of the invention can have etchings or monograms on one or both sides.

Preferred tablets of the invention disintegrate within about 30 to about 300 seconds, more preferably within about 30 to about 200 seconds, and still more preferably within about 30 to about 150 seconds, in a standard in vitro disintegration assay (e.g., conducted according to U.S. Pharmacopeia 24 (2000), Test No. 701).

Alternatively or additionally, preferred tablets of the invention disintegrate within about 5 to about 60 seconds, more preferably within about 5 to about 40 seconds, and still more preferably within about 5 to about 30 seconds, for example within about 25 seconds, after placement in the oral cavity of a subject.

Tablets of the invention have a hardness that can depend on size and shape as well as on composition, among other characteristics. Tablet hardness can be measured by any method known in the art, for example by a tablet hardness meter (e.g., Schleuniger). Preferably, compositions of the invention have a hardness of about 1 to about 10 kP, and more preferably of about 1 to about 5 kP.

In a presently preferred embodiment, solid dosage forms of the invention have sufficient hardness for handling and, therefore, can be put into practical use in the same manner as in the case of conventional tablets. The term “sufficient hardness for handling” as used herein means a hardness which withstands removal from at least a standard type of blister packaging, or other handling such as packaging, delivery, carrying and the like.

Tablets of the invention preferably have a minimum hardness so as to resist breakage of the tablet during removal from standard blister packaging by pushing the tablet through a cover sheet. A suitable hardness is about 1 kP or more for a tablet having a diameter of about 8 mm, about 1.5 kP or more for a tablet having a diameter of about 10 mm, and about 2 kP or more when the tablet has a diameter of about 12 mm.

In another presently preferred embodiment, tablets of the invention have sufficient hardness such that a plurality of such tablets can be packaged together, for example in a glass or plastic bottle, without individual packaging, yet do not exhibit substantial breakage or sticking and/or melding together during normal shipping and handling. Tablets intended for such packaging preferably have a hardness of about 3 kP or more.

Tablets of the invention can be packaged in any suitable manner known in the art. A multiplicity of fast-melt tablets can be packaged together, for example in a glass or plastic bottle or container. Alternatively, fast-melt tablets of the invention can be individually wrapped, for example in plastic or foil, or packaged in known forms of blister packaging. Blister packaging with improved force distribution properties such as is disclosed in U.S. Pat. No. 5,954,204 to Grabowski can be especially useful to package fast-melt tablets of the invention.

Illustrative examples of processes for preparing a fast-melt formulation of the invention are presented with greater particularity below. A compound of formula (II) or salt thereof is illustratively shown as the agent effective in treatment of sexual dysfunction, but any such agent contemplated herein can be substituted.

In one particular embodiment of the invention, the formulation is a porous tablet prepared by providing a mix comprising an inert readily volatilizable solid adjuvant, for example urethane, urea, ammonium carbonate, ammonium bicarbonate, hexamethylenetetramine, benzoic acid, phthalic anhydride, naphthalene, camphor, etc.; compressing the mix to form a tablet; and thereafter volatilizing the adjuvant to form a porous tablet substantially as disclosed in above-cited U.S. Pat. No. 3,885,026. The compound of formula (II) or salt thereof and other desired excipients are present in the mix.

In another particular embodiment of the invention, the formulation is a porous tablet prepared by forming a mixture of the tablet components with a solvent, for example water, cyclohexane, etc., which is inert towards the tablet components and which has a freezing point of about −30° C. to about 25° C., the solvent constituting about 5% to about 80% by weight of the mixture; solidifying the mixture by introduction into an inert cooling medium; compressing the mixture at a temperature below the freezing point of the solvent to form a tablet; and thereafter evaporating the solvent to form a porous tablet substantially as disclosed in above-cited U.S. Pat. No. 4,134,943. The compound of formula (II) or salt thereof and other desired excipients are present in the mix.

In another particular embodiment of the invention, the formulation is a tablet formed by placing in a mold a composition comprising or consisting essentially of the compound of formula (II) or salt thereof and a solution of a water-soluble or water-dispersible carrier material, for example a polypeptide such as partially hydrolyzed gelatin or a polysaccharide such as hydrolyzed dextran, dextrin, sodium alginate, etc., alone or in mixture with other carrier materials such as polyvinyl alcohol, polyvinylpyrrolidone, acacia, etc., in a solvent, preferably water; and subliming, for example by freeze-drying, the solvent to form a tablet within the mold substantially as disclosed in above-cited U.S. Pat. No. 4,371,516. The mold can be a depression in a filmic material suitable as packaging material for the tablet, and a peelable cover sheet can thereafter be adhered to the filmic material, thereby covering the tablet, substantially as disclosed in above-cited U.S. Pat. No. 4,305,502.

In a related embodiment, the process comprises suspending the compound of formula (II) or salt thereof in a melted triglyceride vehicle; spray-congealing the resulting suspension to form discrete solid particles having the drug encapsulated therein; mixing the drug-containing particles with a water-soluble but ethanol-insoluble carbohydrate, for example fructose, dextrose, lactose, sucrose, etc., and a solvent, for example a water-ethanol mixture, to form a damp mass; compressing the damp mass in a mold to form a tablet; and removing the solvent by drying, substantially as disclosed in above-cited U.S. Pat. No. 5,082,667.

In another particular embodiment of the invention, the formulation is a rapidly water-disintegratable tablet comprising a compound of formula (II) or a salt thereof, and having distributed therewithin a small but effective amount of a tablet disintegrating system comprising an unreacted, intimate mixture of alginic acid and a water-soluble metal carbonate in proportions reactive to form alginic acid salt and carbonic acid when the tablet is placed in water, substantially as disclosed in above-cited U.S. Pat. No. 4,414,198.

In another particular embodiment of the invention, the formulation comprises a mass of spun fibers of a readily water-soluble material, for example a sugar such as sucrose, fructose, dextrose, mannitol, sorbitol, lactose, maltose, etc. or a cellulosic material such as methylcellulose, ethylcellulose, hydroxymethylcellulose, hydroxyethylcellulose, alkali metal salts of carboxymethylcellulose, etc., having the compound of formula (II) or salt thereof distributed on or incorporated in the mass of spun fibers, substantially as disclosed in above-cited U.S. Pat. No. 4,855,326. The mass of spun fibers can be formed as in a process for producing cotton candy. This is a melt extrusion process wherein a stock material comprising the water-soluble material, having the compound of formula (II) or salt thereof dispersed therein, is melted and forced through spinnerets. The resulting cotton candy-like material can be lightly compacted to form the formulation of the invention.

In a related embodiment, the formulation is prepared by a process comprising preparing a shearform matrix, for example by a flash-flow process, from a feedstock comprising a saccharide component, for example sucrose optionally mixed with other saccharides such as dextrose, sorbitol, mannitol, etc., optionally with a crystallization enhancer such as a surfactant; adding a crystallization/binding promoter such as an alcohol, e.g., ethanol, polyvinylpyrrolidone or a mixture thereof, and the compound of formula (II) or salt thereof to the shearform matrix to at least partially crystallize the shearform matrix; and thereafter compacting the shearform matrix to form a formulation substantially as disclosed in above-cited U.S. Pat. Nos. 5,587,172 and No. 5,869,098.

In a further related embodiment, the formulation is prepared by a process comprising mixing the compound of formula (II) or salt thereof with a shearform matrix, compacting the resulting mixture in a mold, and curing the compacted mixture by subjecting to environmental conditions of heat, moisture and pressure that induce crystallization to produce a formulation substantially as disclosed in above-cited U.S. Pat. No. 5,622,719.

In any embodiment of the invention having a shearform matrix or matrix of spun fibers, the compound of formula (II) or salt thereof can optionally be formulated using known controlled-release, delayed-release or sustained-release delivery systems, substantially as disclosed in above-cited U.S. Pat. No. 5,518,730 or U.S. Pat. No. 5,733,577. However, for the present purpose it is generally preferred that the compound of formula (II) or salt thereof be in immediate release form to provide rapid onset of the therapeutic or sexual-stimulatory effect.

Suitable apparatus for preparing formulations from a shearform matrix has been disclosed, for example in above-cited U.S. Pat. Nos. 5,653,926 and 5,662,849.

In another particular embodiment of the invention, the formulation comprises an open matrix network having the compound of formula (II) or salt thereof distributed therein, the open matrix network being formed from mannitol in admixture with a gum, for example acacia, guar gum, xanthan gum, tragacanth gum, locust bean gum, pectin, algin, agar, carrageenan, gum arabic, etc., substantially as disclosed in above-cited U.S. Pat. No. 4,946,684.

In another particular embodiment of the invention, the formulation is a tablet comprising a directly compressible solid excipient, typically a sugar, for example sucrose, lactose or sorbitol; a lubricant, preferably a water-soluble lubricant such as sodium dodecyl sulfate; and the compound of formula (II) or salt thereof; and is prepared by mixing the ingredients to form a mixture and directly compressing the mixture to form a tablet substantially as disclosed in above-cited U.S. Pat. No. 5,073,374.

In another particular embodiment of the invention, the formulation is a tablet comprising a water- or saliva-activated effervescent disintegration agent and microparticles containing the compound of formula (II) or salt thereof, substantially as disclosed in above-cited U.S. Pat. No. 5,178,878. In a related embodiment, a particulate effervescent couple is intimately mixed with the compound of formula (II) or salt thereof, each particle of the effervescent couple comprising a solid core of an edible acid and a coating of an edible base in amounts such that upon reaction of the acid and the base a portion of free unreacted acid remains, substantially as disclosed in above-cited U.S. Pat. No. 5,503,846.

In another particular embodiment of the invention, the formulation is a tablet formed by preparing a mixture comprising the compound of formula (II) or salt thereof and a matrix that comprises a gum, for example acacia, guar gum, xanthan gum, tragacanth gum, etc., a carbohydrate base, for example mannitol, dextrose, sucrose, lactose, maltose, maltodextrin, corn syrup solids, etc., and a solvent; shaping the mixture to form a tablet; freezing the mixture; and vacuum drying the frozen mixture above the collapse temperature of the mixture to form a partially collapsed matrix network substantially as disclosed in above-cited U.S. Pat. No. 5,298,261. The “collapse temperature” is the initial melting point or eutectic temperature of the matrix.

In another particular embodiment of the invention, the formulation is a compact tablet at least 50% by weight of which is the compound of formula (II) or salt thereof, and having as inactive ingredients at least one cellulose and/or cellulose derivative, at least one soluble sugar alcohol, at least one sweetener and at least one flavoring agent, substantially as disclosed in above-cited U.S. Pat. No. 5,401,514.

In another particular embodiment of the invention, the formulation is a tablet comprising the compound of formula (II) or salt thereof in a form of coated or non-coated microcrystals or microgranules, and a mixture of excipients comprising a disintegrating agent, preferably a carboxymethylcellulose or an insoluble reticulated polyvinylpyrrolidone, a swelling agent, preferably a starch, a modified starch such as a carboxymethylated starch or a microcrystalline cellulose, and optionally a direct compression sugar such as dextrose, substantially as disclosed in above-cited U.S. Pat. No. 5,464,632.

In another particular embodiment of the invention, the formulation is prepared by a process comprising suspending the compound of formula (II) or salt thereof and a sugar comprising lactose and/or mannitol in a 0.3% to 2% by weight aqueous solution of agar used in an amount of 40% to 60% by weight based on the solid components to form a suspension; filling the suspension into a mold and permitting it to set therein to form a jelly; and thereafter drying the jelly to produce the formulation, substantially as disclosed in above-cited U.S. Pat. No. 5,466,464.

In another particular embodiment of the invention, the formulation is a tablet prepared by a process comprising mixing the compound of formula (II) or salt thereof, a carbohydrate, for example sucrose, starch sugars, sugar alcohols, tetroses, etc., and a barely sufficient amount of water to wet the surface of particles of the carbohydrate, to form a compressible composition, and compression molding the composition to form a tablet substantially as disclosed in above-cited U.S. Pat. No. 5,501,861.

In another particular embodiment of the invention, the formulation comprises a compound of formula (II) or a salt thereof dispersed in a matrix comprising a saccharide of low moldability and a saccharide of high moldability, substantially as disclosed in above-cited U.S. Pat. No. 5,576,014. A preferred process for preparing such a formulation comprises (a) a step of wet granulating the compound of formula (II) or salt thereof together with a binding agent comprising a saccharide of high moldability, and (b) a step of blending with the compound of formula (II) or salt thereof a saccharide of low moldability, wherein the above steps (a) and (b) occur in any order or simultaneously to result in formation of granules. More preferably the process further comprises (c) a step of compressing the granules prepared by any of the processes summarized above to produce a tablet.

In another particular embodiment of the invention, the formulation is a tablet prepared by a process comprising providing an aqueous composition that comprises (a) an aqueous medium, (b) a support agent comprising a polymeric component, for example a non-hydrolyzed gelatin, capable of maintaining a net charge, a solubilizing component, for example a hydrolyzed gelatin, more water-soluble than the polymeric component and capable of maintaining a net charge of the same sign as the polymeric component, and a bulking agent, (c) a volatilizing agent, for example an alcohol, and (d) a buffering agent; drying the aqueous composition, for example by spray drying, to form a particulate support matrix; adding the compound of formula (II) or salt thereof to the particulate support matrix; and compacting the resulting mixture to form a tablet substantially as disclosed in above-cited U.S. Pat. No. 5,587,180 or U.S. Pat. No. 5,807,576.

In another particular embodiment of the invention, the formulation comprises an orally disintegrating delivery system, for example an effervescent delivery system, having incorporated therein microparticles each having a core comprising the compound of formula (II) or salt thereof and a compound which is sweet in taste and has a negative heat of solution, for example mannitol, and a coating comprising a film-forming polymer such as ethylcellulose, substantially as disclosed in above-cited U.S. Pat. No. 5,607,697.

In another particular embodiment of the invention, the formulation is a tablet prepared by a process comprising adding a volatile salt to the compound of formula (II) or salt thereof, a binder such as trehalose, particularly anhydrous trehalose, an additional binder, and other optional excipients with mixing to form a substantially homogeneous mixture; and compressing the mixture to form a tablet substantially as disclosed in above-cited U.S. Pat. No. 5,762,961.

In another particular embodiment of the invention, the formulation is a tablet prepared by kneading a mixture of the compound of formula (II) or salt thereof and a readily water-soluble crystalline or powdery solid, preferably one having a sweet taste such as sucrose, lactose, glucose, fructose, xylitol, sorbitol, mannitol, etc., with a suitable amount of water, typically about 1% to about 10% by weight of the tablet components; compressively shaping the resulting wet kneaded mixture to form a tablet; and thereafter drying the tablet substantially as disclosed in above-cited U.S. Pat. No. 5,837,285.

In another particular embodiment of the invention, the formulation is a tablet or wafer prepared by a process comprising coating particles of the compound of formula (II) or salt thereof with a taste-masking composition, preferably one that comprises a first polymer selected from cellulose acetate and cellulose acetate butyrate and a second polymer selected from polyvinylpyrrolidone and hydroxypropylcellulose in a weight ratio of first polymer to second polymer of about 90:10 to about 50:50; dry-blending the coated particles with a compressible carbohydrate, for example mannitol, sorbitol, dextrose, sucrose, xylitol, lactose, etc., and a binder, for example cellulose (in particular microcrystalline cellulose), cellulosic derivatives, polyvinylpyrrolidone, starch, modified starch, etc.; and compressing the resulting dry blend to form a tablet or wafer substantially as disclosed in above-cited U.S. Pat. No. 5,876,759.

In another particular embodiment of the invention, the formulation is a tablet prepared by a process comprising a step of preparing compact granules by pre-compacting by mechanical means, for example rolling, or by spray-drying a feedstock comprising a carbohydrate, for example a saccharide of low or high moldability such as maltose, maltitol, sorbitol, mannitol, glucose, sucrose, xylitol, etc., and optionally a low density alkaline-earth metal salt; and a step of compressing the compact granules, optionally with other ingredients, to prepare the tablet substantially as disclosed in above-cited U.S. Pat. No. 5,939,091. A compound of formula (II) or a salt thereof can be added at any suitable stage in the process to result in a fast-melt formulation of the present invention.

In another particular embodiment of the invention, the formulation is a tablet prepared by a process comprising mixing the compound of formula (II) or salt thereof and a carrier that comprises one or more carbohydrates and a binder to form a blend; kneading the blend with about 1% to about 10% by weight of water; drying the kneaded blend and milling to form a compressible powder; and compressing the powder to form a tablet. The carbohydrates can include saccharides and starches, for example erythritol and microcrystalline cellulose substantially as disclosed in above-cited U.S. Pat. No. 5,958,453.

In another particular embodiment of the invention, the formulation is a tablet prepared by freeze-drying and comprising the compound of formula (II) or salt thereof, a matrix forming agent such as a maltodextrin having a dextrose equivalent value of about 12 to about 40 or isomalt, and a binding agent, substantially as disclosed in above-cited U.S. Pat. No. 6,010,719.

In another particular embodiment of the invention, the formulation is a tablet prepared by direct compression and comprising the compound of formula (II) or salt thereof, a non-direct compression filler, preferably a non-direct compression sugar or sugar alcohol such as dextrose, mannitol, sorbitol, lactose, sucrose, etc., and a lubricant, substantially as disclosed in above-cited U.S. Pat. No. 6,024,981.

Fast-melt tablets of the invention can be taken by a subject by any oral administration means in accordance with the subject's choice or condition. For example, fast-melt tablets can be taken without water. Upon placement in the oral cavity and especially in the cheek or above the tongue, such a tablet is exposed to saliva and rapidly disintegrates therein. The rate of disintegration increases further when an intraoral pressure, for example a pressure between the palate and tongue or a licking or sucking pressure, is applied to the tablet.

Alternatively, a fast-melt tablet of the invention can be taken with the aid of water in an amount sufficient to wet the oral cavity and to assist in disintegration of the tablet. Also, a fast-melt tablet can be swallowed together with a small amount of water after complete or partial disintegration in the oral cavity. Fast-melt tablets of the invention can also be swallowed directly with water.

Preferably, a dosage form of the invention is therapeutically effective when administered less than about 1 hour, more preferably less than 30 minutes, prior to sexual activity. Most preferred dosage forms of the invention are therapeutically effective when administered about 5 minutes to about 20 minutes, for example about 10 minutes to about 15 minutes, prior to sexual activity.

### EXAMPLES

The following examples illustrate aspects of the present invention but should not be construed as limitations. In these examples “compound Z” refers to (R)-5,6-dihydro-5-(methylamino)-4H-imidazo[4,5-ij]-quinoline-2(1H)-thione, maleate salt. All percentages are by weight unless otherwise indicated.

### Example 1

A sublingual tablet formulation was prepared
having the following composition:

| | |
|---|---|
| compound Z | 1.11% |
| Avicel ™ PH-101 (microcrystalline cellulose) | 46.71% |
| Starch 1500 of Colorcon (pregelatinized starch) | 44.00% |
| croscarmellose sodium NF | 5.00% |
| colloidal silicon dioxide NF | 0.50% |
| cinnamon flavor | 0.14% |
| mint flavor | 0.04% |
| color (cherry shade #1632, Crompton & Knowles) | 0.50% |
| magnesium stearate | 2.00% |

Pregelatinized starch and color were blended in a high-shear mixer for 2 minutes or until homogeneously mixed. The following ingredients were then individually layered over the resulting mixture in the high-shear mixer: compound Z; microcrystalline cellulose; colloidal silicon dioxide; croscarmellose sodium. Mixing in the high-shear mixer was resumed for a further 2 minutes. If the color was not adequately dispersed throughout the resulting mixture, mixing continued in 1 minute increments until good dispersion of color was observed. A small portion of the mixture was then removed and hand-mixed with magnesium stearate to form a magnesium stearate premix. This premix, together with the flavors, was added to the high-shear mixer and mixed for 1 minute to form a lubricated tablet stock.

The lubricated tablet stock was discharged from the high-shear mixer and stored in desiccated hermetically sealed containers until ready for tableting. Tablets were prepared by compression using 12/32 inch (approximately 9 mm) Plain/Plain tooling with slight curvature to the following specifications:

| | | |
|---|---|---|
| tablet weight | 180 | mg |
| hardness | 3-4 | SCU |
| friability | <0.5% | |

### Example 2

Sublingual tablets prepared as in Example 1 were coated with a gellan gum coating according to the following procedure.

A coating liquid having the following composition was prepared:

| | |
|---|---|
| gellan gum (Kelcogel ™) | 2.00% |
| sodium citrate | 0.13% |
| propylene glycol | 0.40% |
| lecithin | 0.20% |
| deionized water | 97.27% |

Deionized water was heated to 70° C.. The other ingredients were added with stirring until all ingredients were homogeneously dispersed. The resulting coating liquid having a solids content of 2.73% was maintained at a temperature of 70° C. during the stirring and subsequent spraying procedure.

Tablets of Example 1, in an amount of 700 g, were placed in a 12 inch (approximately 300 mm) coating pan and preheated to a bed temperature of 60° C. The coating liquid was sprayed on to the tablets under the following conditions:

| | | |
|---|---|---|
| outlet air temperature | 50-60° | C. |
| pan speed | 16 | rpm |
| air flow | 30-35 | cfm (0.84-0.98 m³/minute) |
| atomizing air pressure | 10 | psi (69 kPa) |
| peristaltic pump setting | 15-20 | g/minute |

Spraying was continued until an amount of coating solution equivalent to a weight gain of 1.2% had been applied. The resulting coated tablets were cooled to 30° C. prior to discharge from the coating pan.

### Example 3

A sublingual tablet formulation was prepared
having the following composition:

| | |
|---|---|
| compound Z | 1.05% |
| mannitol, granular | 70.00% |
| sorbitol | 16.57% |
| hydroxypropylcellulose, type LH-11 | 7.00% |
| xanthan gum | 2.50% |
| colloidal silicon dioxide NF | 0.50% |
| cinnamon flavor | 0.14% |
| mint flavor | 0.04% |
| color (cherry shade #1632, Crompton & Knowles) | 0.20% |
| magnesium stearate | 2.00% |

Mannitol and color were blended in a high-shear mixer for 2 minutes or until homogeneously mixed. The following ingredients were then individually layered over the resulting mixture in the high-shear mixer: compound Z; sorbitol; hydroxypropylcellulose; xanthan gum; colloidal silicon dioxide. Mixing in the high-shear mixer was resumed for a further 2 minutes. If the color was not adequately dispersed throughout the resulting mixture, mixing continued in 1 minute increments until good dispersion of color was observed. A small portion of the mixture was then removed and hand-mixed with magnesium stearate to form a magnesium stearate premix. This premix, together with the flavors, was added to the high-shear mixer and mixed for 1 minute to form a lubricated tablet stock.

The lubricated tablet stock was discharged from the high-shear mixer and stored in desiccated hermetically sealed containers until ready for tableting. Tablets were prepared by compression using 12/32 inch (approximately 9 mm) Plain/Plain tooling with slight curvature to the following specifications:

| | | |
|---|---|---|
| tablet weight | 190 | mg |
| hardness | 3-4 | SCU |
| friability | <0.5% | |

### Example 4

Sublingual tablets prepared as in Example 3 were coated with a gellan gum coating according to the following procedure.

A coating liquid having the following composition was prepared:

| | |
|---|---|
| gellan gum (Kelcogel ™) | 2.00% |
| sodium citrate | 0.13% |
| propylene glycol | 0.40% |
| lecithin (Lipoid ™ LS-100) | 0.20% |
| flavor | 0.30% |
| deionized water | 96.97% |

Deionized water was heated to 70° C. The other ingredients were added with stirring until all ingredients were homogeneously dispersed. The resulting coating liquid having a solids content of 3.03% was maintained at a temperature of 70° C. during the stirring and subsequent spraying procedure.

Tablets of Example 1, in an amount of 700 g, were placed in a 12 inch (approximately 300 mm) coating pan and preheated to a bed temperature of 60° C. The coating liquid was sprayed on to the tablets under the following conditions:

| | | |
|---|---|---|
| outlet air temperature | 50-60° | C. |
| pan speed | 16 | rpm |
| air flow | 30-35 | cfm (0.84-0.98 m³/minute) |
| atomizing air pressure | 10 | psi (69 kPa) |
| peristaltic pump setting | 15-20 | g/minute |

Spraying was continued until an amount of coating solution equivalent to a weight gain of 1.36% had been applied. The resulting coated tablets were cooled to 30° C. prior to discharge from the coating pan.

What is claimed is:

1. A pharmaceutical dosage form comprising (a) at least one agent effective in treatment of sexual dysfunction having a molecular weight, excluding counterions, not greater than 250, in a therapeutically or sexual-stimulatorily effective total amount, and (b) at least one pharmaceutically acceptable excipient; the dosage form being adapted for delivery by a route of administration that entails interaction with the organs of taste yet having acceptable organoleptic properties.

2. The dosage form of claim 1 wherein the at least one agent has a molecular weight, excluding counterions, not greater than 235.

3. The dosage form of claim 1 wherein the at least one agent has a molecular weight, excluding counterions, not greater than 220.

4. The dosage form of claim 1 wherein the at least one agent has a solubility in water at 20-25° C. of at least about 10 g/l.

5. The dosage form of claim 1 wherein the at least one agent is a compound having the formula 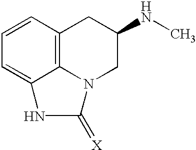
wherein X is O or S; or a pharmaceutically acceptable salt thereof.

6. The dosage form of claim 1 wherein the total amount of the at least one agent per dose is lower than an amount causing significant side-effects.

7. The dosage form of claim 1 wherein the therapeutic agent is sumanirole or a salt thereof and is present in an amount of about 0.05 mg to about 5 mg per dose.

8. The dosage form of claim 1 wherein the therapeutic agent is (R)-5,6-dihydro-5-(methylamino)-4H-imidazo[4,5-ij]-quinoline-2(1H)-thione or a salt thereof and is present in an amount of about 0.05 mg to about 5 mg per dose.

9. The dosage form of claim 8 wherein the therapeutic agent is present in an amount of about 0.1 to about 3 mg per dose.

10. The dosage form of claim 1 that is adapted for a route of administration selected from oral, buccal, sublingual, nasal and tracheal routes.

11. The dosage form of claim 1 that is selected from
(a) buccal and sublingual tablets;
(b) mucoadhesive films;
(c) oral strips;
(d) chewable tablets;
(e) rapidly disintegrating oral dosage forms;
(f) lozenges and pastilles;
(g) breath-fresheners;
(h) chewing gums;
(i) lollipops and popsicles;
(j) food adjuncts;
(k) candies and chocolates;
(l) periodontal gels;
(m) mouthwashes;
(n) oral and nasal drops and sprays;
(o) dosage forms adapted for inhalation as an aerosol or vapor;
(p) elixirs, solutions, suspensions and other orally administered liquid dosage forms;
(q) powders, granules and tablets for dissolution or dispersion in water prior to oral administration; and
(r) effervescent tablets and granules.

12. The dosage form of claim 1 that is adapted for discreet self-administration.

13. The dosage form of claim 1 that is adapted for nasal administration.

14. The dosage form of claim 13 that is formulated as a nasal spray solution.

15. The dosage form of claim 1 that is adapted for oral, buccal or sublingual administration.

16. The dosage form of claim 15 that dissolves in the mouth without need for drinking water or other fluid.

17. The dosage form of claim 15 that is a breath-freshening pastille.

18. The dosage form of claim 15 that is a chewing gum.

19. The dosage form of claim 15 that is a sublingual tablet.

20. The dosage form of claim 15 that is a mucoadhesive film.

21. The dosage form of claim 15 that is an oral strip.

22. The dosage form of claim 15 that is an oral fast-melt tablet.

23. A pharmaceutical dosage form comprising (a) a therapeutically or sexual-stimulatorily effective amount of about 0.1 mg to about 10 mg per dose of a therapeutic agent that comprises at least one compound of formula 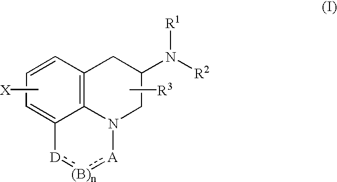
or a pharmaceutically acceptable water-soluble salt thereof, said compound or salt thereof being water-soluble, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃;
and (b) one or more pharmaceutically acceptable excipients; the dosage form being adapted for delivery by a route of administration that entails interaction with the organs of taste yet having acceptable organoleptic properties.

24. The dosage form of claim 23 wherein the water-soluble compound or salt thereof has a solubility in water at 20-25° C. of at least about 10 g/l.

25. The dosage form of claim 23 wherein the water-soluble compound or salt thereof is disclosed generically or specifically in U.S. Pat. No. 5,273,975.

26. The dosage form of claim 23 that is adapted for a route of administration selected from oral, buccal, sublingual, nasal and tracheal routes.

27. The dosage form of claim 23 that is selected from
(a) buccal and sublingual tablets;
(b) mucoadhesive films;
(c) oral strips;
(d) chewable tablets;
(e) rapidly disintegrating oral dosage forms;
(f) lozenges and pastilles;
(g) breath-fresheners;
(h) chewing gums;
(i) lollipops and popsicles;
(j) food adjuncts;
(k) candies and chocolates;
(l) periodontal gels;
(m) mouthwashes;
(n) oral and nasal drops and sprays;
(o) dosage forms adapted for inhalation as an aerosol or vapor;
(p) elixirs, solutions, suspensions and other orally administered liquid dosage forms;
(q) powders, granules and tablets for dissolution or dispersion in water prior to oral administration; and
(r) effervescent tablets and granules.

28. The dosage form of claim 23 that is adapted for discreet self-administration.

29. The dosage form of claim 23 that is adapted for nasal administration.

30. The dosage form of claim 29 that is formulated as a nasal spray solution.

31. The dosage form of claim 23 that is adapted for oral, buccal or sublingual administration.

32. The dosage form of claim 31 that dissolves in the mouth without need for drinking water or other fluid.

33. The dosage form of claim 31 that is a breath-freshening pastille.

34. The dosage form of claim 31 that is a chewing gum.

35. The dosage form of claim 31 that is a sublingual tablet.

36. The dosage form of claim 31 that is a mucoadhesive film.

37. The dosage form of claim 31 that is an oral strip.

38. The dosage form of claim 31 that is an oral fast-melt tablet.

39. A method of treating sexual dysfunction in a subject comprising intraoral administration of a dosage form of claim 1 to the subject, less than about 1 hour prior to sexual activity.

40. A method of treating sexual dysfunction in a subject comprising intraoral administration of a dosage form of claim 23 to the subject, less than about 1 hour prior to sexual activity.

41. A method of enhancing sexual desire, interest or performance in a subject comprising intraoral administration of a dosage form of claim 1 to the subject, less than about 1 hour prior to sexual activity.

42. A method of enhancing sexual desire, interest or performance in a subject comprising intraoral administration of a dosage form of claim 23 to the subject, less than about 1 hour prior to sexual activity.
